(19) United States
(12) Patent Application Publication
(10) Pub. No.: US 2019/0023811 A1
(43) Pub. Date: Jan. 24, 2019

(54) DRY BIOCELLULOSE MATERIAL AND PREPARATION THEREOF

(71) Applicant: TCI CO., LTD., Taipei (TW)

(72) Inventors: Yung-Hsiang Lin, Taipei (TW); Cheng-Yu Ho, Taipei (TW); Wei-Hsiu Chuang, Taipei (TW)

(21) Appl. No.: 16/039,630

(22) Filed: Jul. 19, 2018

(30) Foreign Application Priority Data
Jul. 21, 2017 (TW) 106124592

(51) Int. Cl. C08B 1/00; A61K 8/02; A61K 8/73; A45D 44/00

(52) U.S. Cl. CPC C08B 1/003; A45D 44/002; A61K 8/731; A61K 8/0212

(57) ABSTRACT

The present invention provides a method of preparing a dry biocellulose material, including immersing a biocellulose material into an aqueous solution containing 10% or a higher concentration of glycerol for a predetermined time, and drying the biocellulose material containing glycerol. The present invention also provides a dry biocellulose material prepared thereby. The preparation method of the present invention prevents irreversible impairment to the fibrous structure of the biocellulose material due to drying at high temperatures. Thus, the dry biocellulose material of the present invention may quickly regain a condition of high water content after contact with water.

### CROSS-REFERENCE TO RELATED APPLICATION

This application claims priority of Taiwan application No. 106124592, filed on Jul. 21, 2017 the content of which is incorporated herein in its entirety by reference.

### BACKGROUND OF THE INVENTION

### 1. Field of the Invention

The present invention relates to a biocellulose material and a preparation method thereof, and particularly to a dry biocellulose material can quickly regain a condition of high water content after contact with water and a preparation method thereof.

### 2. The Prior Art

Biocellulose, known as bacterial cellulose or microbial cellulose, is cellulose produced by bacteria. Its essence is a linear polysaccharide formed by linking glucose with β-1,4 glycosidic bonds. In the industry uses, biocellulose are commonly produced by Acetobacter xylinum. Compared to plant fibers, biocellulose are finer in diameter (about 25 nm-100 nm) and have a tighter structure. The chemical composition is purer than unprocessed plant fibers because plant fibers are usually mixed with hemicellulose and lignin. In addition, compared with general plant fibers, the tension, the plasticity, and the water content of biocellulose are rich, better, and higher. Thus, the biocellulose is more suitable for use in biomedical materials such as wound dressings, artificial skin, and masks.

Commercially available biocellulose products, such as biocellulose masks, are mostly present in an aqueous form. However, aqueous biocellulose are prone to bacteria or molds, so such products often require the addition of bacteriostats or preservatives. Furthermore, large spaces are required for transportation and storage since the volume of water-containing biocellulose is large, and the shipping and storage costs are increased. In order to solve the aforementioned problems, the researchers tried to make dry biocellulose materials, but found that once the biocellulose were dried, the overlapping fiber structures were destroyed, so that the formed pores were tight, even if the dried biocellulose is contacted with water again, the fiber structure is still difficult to recover, resulting in a significant reduction in the water content capacity of the dry biocellulose material.

In view of the above, it is necessary to develop a novel method for preparing a dry biocellulose material, so that to make the dry biocellulose material can quickly regain a condition of high water content after contacting with water or an aqueous solution.

### SUMMARY OF THE INVENTION

To solve the foregoing problem, one objective of the present invention is to provide a method of preparing a dry biocellulose material, comprising the steps of: (i) immersing a biocellulose material in a glycerin solution for a predetermined period of time to form a glycerin-containing biocellulose material; and (ii) drying the glycerin-containing biocellulose material to obtain a dry biocellulose material; wherein the glycerin solution contains glycerol with a concentration of 10% or more.

In one embodiment of the present invention, the predetermined period of time is 30-60 minutes; and the drying process is performed by a drier at a temperature from 80° C. to 85° C. for 2-4 hours.

In another aspect, the present invention provides a dry biocellulose material, wherein the dry biocellulose material is produced by the method according to the foregoing preparation.

In another aspect, the present invention provides a dry biocellulose mask, which is obtained by processing the dry biocellulose material, having a shape corresponding to an entire face of a subject, and includes multiple pre-cut openings in corresponding eyes, a nose, and a mouth of the subject.

In one embodiment of the present invention, the dry biocellulose mask contacts with water or an aqueous solution for 10 minutes to restore a rehydration state before the drying process and the weight of the dry biocellulose mask after contacting with water or the aqueous solution for 10 minutes is 10-20 times heavier than the weight of the dry biocellulose mask.

The method of the present invention is to immerse the biocellulose material in a glycerin solution with the concentration of 10% or more to prevent the irreversible impairment to fiber structure due to drying treatment; therefore, the dry biocellulose material can be restored to the high water-containing state after re-immersing in the water. Compared with the conventional biocellulose material, the drying process would impair the fiber structure of the biocellulose material, and the preparation method of the present invention prevents irreversible impairment to the fibrous structure of the biocellulose material due to drying at high temperatures, so that the biocellulose material can quickly regain a condition of high water content after contact with water. At the same time, the dry biocellulose material prepared by the method of the present invention is easier to store than the moist biocellulose material, and requires a small storage space and does not need to be added with a preservative, thereby reducing production and transportation costs.

The present invention is further described in the following examples, in reference to the accompanying drawings. It should be understood that the examples given below do not limit the scope of the invention, and that modifications can be made without departing from the scope of the appended claims.

### BRIEF DESCRIPTION OF THE DRAWINGS

The present invention will be apparent to those skilled in the art from the following detailed description of the preferred embodiments, with reference to the attached drawings, in which:

FIG. 1 shows the pictures of the biocellulose material immersed in the different concentration of the glycerin solution and the biocellulose mask restoring to the rehydration state.

FIG. 2 shows the weight changes of the biocellulose mask immersed in the different concentration of the glycerin solution.

FIG. 3 shows the weight changes of the biocellulose mask, which has been immersed in the different concentration of the glycerin solution, before drying or after restoring to the rehydration state.

FIG. 4 shows the thickness of the biocellulose material immersed in the different concentration of the glycerin solution and the biocellulose mask restoring to the rehydration state.

### DETAILED DESCRIPTION OF THE PREFERRED EMBODIMENT

### Definition

The data provided in the present invention represent approximated, experimental values that may vary within a range of ±20%, preferably ±10%, and most preferably ±5%.

The biocellulose material refers to a membrane composed of biocellulose produced by various microorganisms, such as Acetobacter xylinum, Gluconacetobacter hansenii, or Acetobacter pasteurianus.

The present invention provides a method for preparing a dry biocellulose material, the method comprising: immersing a biocellulose material in a glycerin solution with a concentration of 10% or more for a period of time, and drying the glycerin-containing biocellulose material, such as drying at high temperatures. In the following examples, the preparation method of the dry biocellulose material is exemplified by the biocellulose mask, and to evaluate the rehydration state of the biocellulose mask after immersing in the different concentration of the glycerin solution, drying, and re-immersing in water. The experimental results show that when the concentration of the glycerin solution is more than 10%, the rehydration of the dry biocellulose mask is significantly improved, and its appearance and feel are similar to those of the biocellulose mask without drying process.

### EXAMPLE 1

### Preparation of the Dry Biocellulose Mask

This example illustrates the preparation of the dry biocellulose mask of the present invention. First, an appropriate amount of glycerol is mixed with distilled water to prepare a glycerol solution with a weight percentage of 10% or more. At the same time, the biocellulose material is processed to have a mask appearance, referred to as a biocellulose mask, and rinses with distilled water. The biocellulose mask has a shape corresponding to an entire face of a subject, and includes multiple pre-cut openings in corresponding eyes, a nose, and a mouth of the subject.

Next, the rinsed biocellulose mask is immersed in the glycerin aqueous solution for 30-60 minutes, and the biocellulose mask can be fully contacted with glycerin by extruding, shaking, stirring, etc. during the immersing process. After the immersion is completed, the glycerin-containing biocellulose mask is taken out and dried to no more droping glycerin solution, and then placed in an oven and dried at 80° C.-859° C. for 2-4 hours, for example, dried at 80° C. for 3 hours. Finally, the dry biocellulose mask of the present invention can be obtained. The drying time varies depending on the drying temperature.

### EXAMPLE 2

Evaluating the Effect of the Glycerin Solution Concentration on the Appearance of the Dry Biocellulose Mask after Rehydration

To test whether the dry biocellulose mask prepared by the method of the present invention can quickly return to the normal appearance after rehydration, according to the procedure of EXAMPLE 1, prepare a glycerin solution with a weight percentage of 1%, 2.5%, 5%, 7.5%, or 10%, respectively, and separately immersed the biocellulose mask, which has been pre-rinsed with distilled water, in the different weight percentage of the glycerin solution for 30-60 minutes. After the immersion is completed, each group of glycerin-containing biocellulose masks are dried to no more droping glycerin solution, and then placed in an oven to be dried at 80° C. for 3 hours to obtain dry biocellulose masks with different concentration of glycerol. Thereafter, each of the above-mentioned biocellulose masks is immersed in distilled water for 10 minutes and then taken out to compare the change in the appearance. As a comparison benchmark, a biocellulose mask that is not immersed in a glycerin solution and not dried is used as a control group.

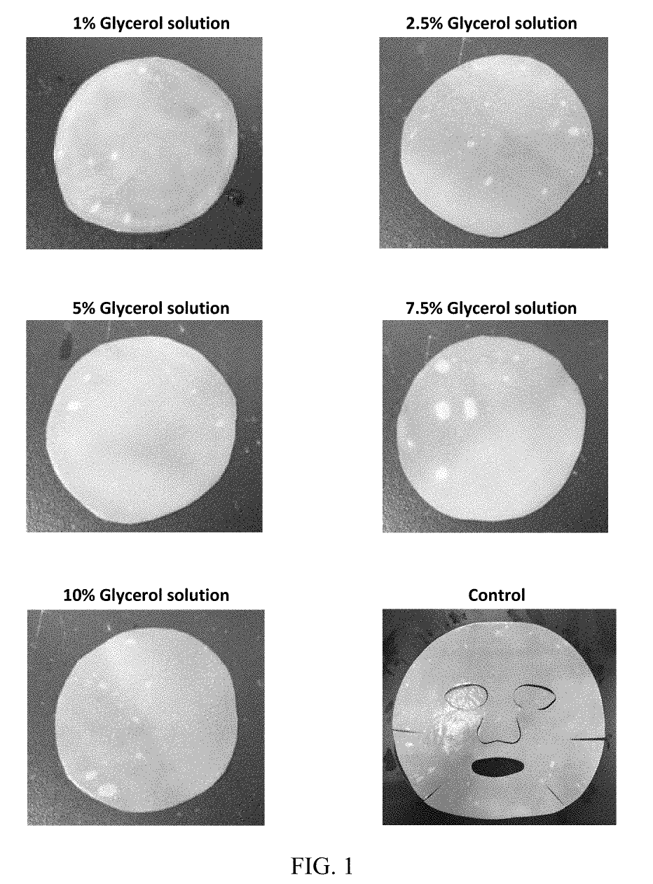

FIG. 1 shows the pictures of the biocellulose material immersed in the different concentration of the glycerin solution and the biocellulose mask of the present invention to rehydrate in distilled water. According to FIG. 1, the biocellulose mask immersed with 1% glycerin solution is hardened after re-immersing, and is not easy to stick to a surface of the subject, and has a plastic touch. In contrast, the biocellulose mask immersed with 2.5%, 5%, or 7.5% glycerin solution and the dry biocellulose mask of the present invention have similar appearance and sense of touch to the control group after re-immersing in water. The result shows that the biocellulose mask immersed in the glycerin solution at concentration of 2.5% or more enables to retain the undestroyed fibrous structure after being subjected to a high temperature treatment at 80-85° C., so the appearance can be restored after rehydration.

### EXAMPLE 3

### The Effect of the Glycerin Solution Concentration on the Rehydration State of the Dry Biocellulose Mask

To test the rehydration effect of the dry biocellulose material prepared by the method of the present invention, according to the procedure of EXAMPLE 1, prepare a glycerin solution with a weight percentage of 1%, 2.5%, 5%, 7.5%, or 10%, respectively, and separately immersed the biocellulose mask, which has been pre-rinsed with distilled water, in the different weight percentage of the glycerin solution for 30-60 minutes, and each group is repeated 3 times. After the immersion is completed, each group of glycerin-containing biocellulose masks are dried to no more droping glycerin solution, and then placed in an oven to be dried at 80° C. for 3 hours to obtain dry biocellulose masks with different concentration of glycerol. Thereafter, each of the above-mentioned biocellulose masks is immersed in distilled water for 10 minutes and then taken out to test the rehydration effect.

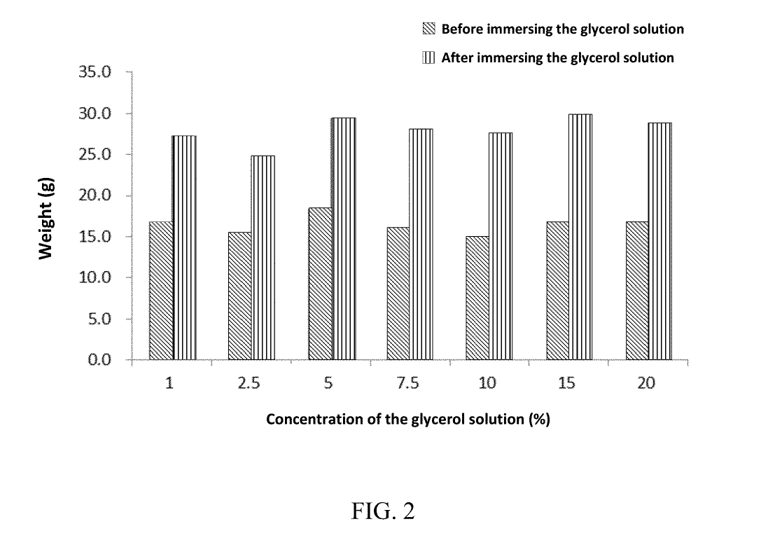

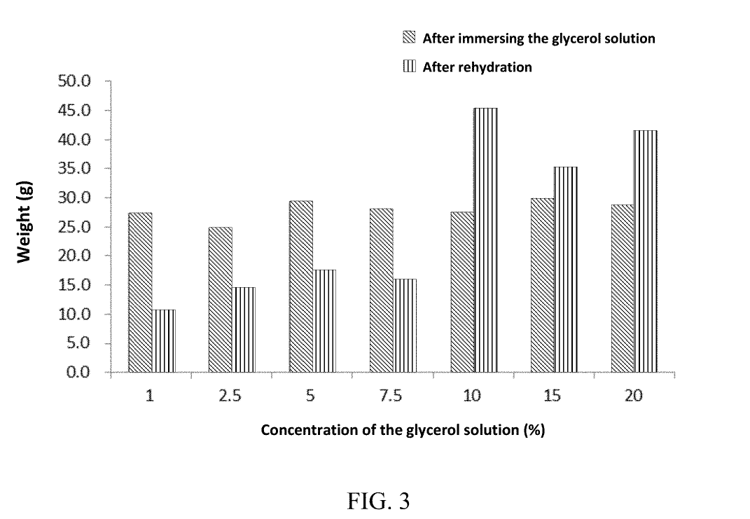

FIG. 2 shows the weight changes of the biocellulose mask immersed in the different concentration of the glycerin solution. FIG. 3 shows the weight changes of the biocellulose mask, which has been immersed in the different concentration of the glycerin solution, before drying or after restoring to the rehydration state. According to FIG. 2, the weight increase of the biocellulose mask is not significantly different due to the difference in the concentration of the glycerin solution immersed, and the average weight increase is 11.5 g/piece.

However, according to FIG. 3, when the concentration of glycerin solution is more than 10% compared to less than 10%, the biocellulose mask has a significantly heavier weight in the water rehydrating test after drying process, and the weight is greater than the weight of a biocellulose mask which is simply immersed in the glycerin solution without drying process. The result indicates that the rehydration effect of the dry biocellulose mask of the present invention is better. Further, the rehydration weight is 10-20 times heavier than the weight of the dry biocellulose mask of the present invention. These results indicate that the biocellulose mask immersed in the glycerin solution at concentration of 10% or more enables to have a strong rehydration capacity after being subjected to a high temperature treatment at 80-85° C. and quickly regain a condition of high water content after contact with water.

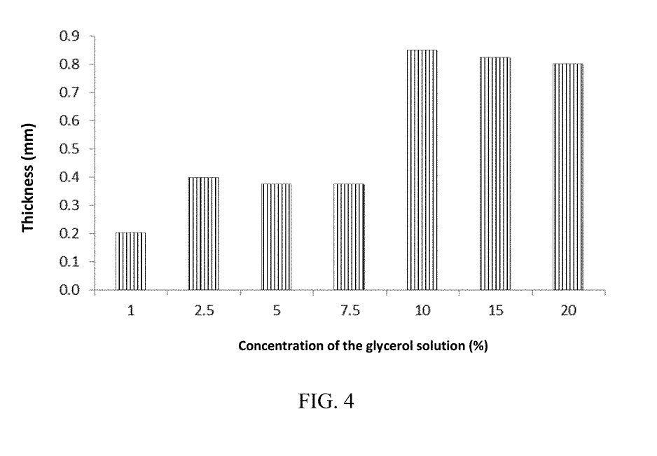

FIG. 4 shows the thickness of the biocellulose material immersed in the different concentration of the glycerin solution and the biocellulose mask restoring to the rehydration state. According to FIG. 4, the dry biocellulose mask immersed with 1% glycerin solution has a thickness of about 0.2 mm after rehydrating; the thickness of the dry biocellulose mask immersed with 2.5%, 5%, or 7.5% glycerin solution is increased to about 0.3-0.4 mm; the thickness of the dry biocellulose mask of the present invention immersed in 10%, 15%, or 20% glycerin is greatly increased to about 0.8 mm or more after rehydrating. Compared with the dry state, the thickness of the dry biocellulose mask of the above each group is about 0.1 mm and the thickness of the dry biocellulose mask of the present invention is about 0.1-0.2 mm The results prove that the method of the present invention can produce a dry biocellulose material with a good rehydration effect.

In summary, the method of the present invention prevents irreversible impairment to the fibrous structure of the biocellulose material due to drying at high temperatures by immersing in glycerin solution with a concentration of 10% or more. Thus, the dry biocellulose material of the present invention may quickly restore the appearance, sense of touch, thickness, and regain a condition of high water content after contact with water. Compared with the conventional process, the drying process without immersing in a glycerol solution would destroy the fiber structure of the biocellulose material. In addition, the dry biocellulose material prepared by the present invention is easier to store than the moist biocellulose material, and requires a small storage space and does not need to be added with a preservative, thereby reducing production and transportation costs, thereby reducing production and transportation costs.

What is claimed is:

1. A method of preparing a dry biocellulose material, comprising the steps of:
(i) immersing a biocellulose material in a glycerin solution for a predetermined period of time to form a glycerin-containing biocellulose material; and
(ii) drying the glycerin-containing biocellulose material to obtain a dry biocellulose material;
wherein the glycerin solution contains glycerol with a concentration of 10% or more.

2. The method according to claim 1, wherein the predetermined period of time is 30-60 minutes.

3. The method according to claim 1, wherein the drying process is performed by a drier.

4. The method according to claim 3, wherein the drying process is performed at a temperature from 80° C. to 85° C.

5. The method according to claim 4, wherein the drying process is performed for 2-4 hours.

6. A dry biocellulose material, which is produced by the method according to claim 1.

7. A dry biocellulose material, which is produced by the method according to claim 2.

8. A dry biocellulose material, which is produced by the method according to claim 3.

9. A dry biocellulose material, which is produced by the method according to claim 4.

10. A dry biocellulose material, which is produced by the method according to claim 5.

11. A dry biocellulose mask, which is obtained by processing the dry biocellulose material according to claim 6, having a shape corresponding to an entire face of a subject, and includes multiple pre-cut openings in corresponding eyes, a nose, and a mouth of the subject.

12. The dry biocellulose mask according to claim 11, wherein the dry biocellulose mask contacts with water or an aqueous solution for 10 minutes to restore a rehydration state before the drying process.

13. The dry biocellulose mask according to claim 11, wherein the weight of the dry biocellulose mask after contacting with water or the aqueous solution for 10 minutes is 109-20 times heavier than the weight of the dry biocellulose mask.
